(19) United States
(12) Patent Application Publication
(10) Pub. No.: US 2022/0168460 A1
(43) Pub. Date: Jun. 2, 2022

(54) AIR PURIFICATION AND PROTECTION SYSTEM

(71) Applicants: Craig H. LUSSI, Gibson Island, MD (US); Hunter M. LUSSI, Gibson Island, MD (US); Tyler T. LUSSI, Gibson Island, MD (US); Morgan M. LUSSI, Gibson Island, MD (US)

(72) Inventors: Craig H. LUSSI, Gibson Island, MD (US); Hunter M. LUSSI, Gibson Island, MD (US); Tyler T. LUSSI, Gibson Island, MD (US); Morgan M. LUSSI, Gibson Island, MD (US)

(21) Appl. No.: 17/538,553

(22) Filed: Nov. 30, 2021

Related U.S. Application Data
(60) Provisional application No. 63/120,193, filed on Dec. 1, 2020.

(51) Int. Cl. A61L 9/20

(52) U.S. Cl. CPC A61L 9/20

(57) ABSTRACT

An incapacitation of biological contaminants in air in interior space and interior air protection system and method is provided. The incapacitation of biological contaminants in air in interior space and interior air protection system and method can be for an interior space and can utilize additional UV radiation exposure of interior air which incapacitates biological contaminants in interior space as they exist, are produced and are transmitted, and can provide a barrier of air moving upwards towards additional UV radiation exposure, a portion of whose biological contaminants have been incapacitated, to inhibit transmission of biological contaminants and provide additional incapacitation of biological contaminants in interior space that is taller than approximately 6.5 to approximately 8.5 feet. The resulting interior air can be free of biological contaminants after passing through the lower and upper air UV radiation.

### CROSS-REFERENCE TO RELATED APPLICATIONS

The present application claims benefit of and priority to U.S. Provisional Patent Application No. 63/120,193, filed Dec. 1, 2020, which is incorporated herein by reference in its entirety for all purposes.

### FIELD

The present disclosure relates to air purifiers and using purified air to protect from biological contaminants (including but not limited to viruses and bacteria). In particular, the present disclosure relates to air purifiers utilizing ultraviolet radiation and purified air to protect against and/or redirect the spread of biological contaminants (including but not limited to viruses and bacteria) to be incapacitated by ultraviolet radiation.

### BACKGROUND

Air purifiers utilizing ultraviolet radiation can be used to incapacitate biological contaminants in the air. In particular, the infection by biological contaminants of living organisms can be reduced or eliminated by using ultraviolet radiation because ultraviolet light induces damage to the genomes of bacteria, protozoa, and viruses. The ultraviolet radiation breaks bonds and forms photodimeric lesions in nucleic acids, DNA, and RNA and these lesions prevent both transcription and replication and ultimately lead to inactivation of the microorganisms.

### BRIEF SUMMARY

Air purifying ultraviolet radiation (UV) can be incorporated into HVAC ductwork, portable units most often placed in lower air, upper air UV, or FAR 222 UV.

Disclosed herein are aspects directed to additional incapacitation of biological contaminants in air that utilize lower and upper UV exposure of air. Upper air UV bulbs installed in upper interior areas in buildings and homes (mounted approximately 6.5 to approximately 8.5 feet high to protect people's skin and eyes) can utilize upper air UV systems in combination with enclosed portable UV air purifiers. The enclosed portable UV air purifiers can be placed in lower air areas with directional exhaust air to direct air to upper air UV areas so the biological contaminants (including but not limited to viruses and bacteria) are incapacitated in upper air UV after being discharged from people's bodies. These units can be utilized in schools, offices, shopping centers, sports venues, dining areas, and other enclosed social settings. Ultraviolet radiation, for example, beamed by UV systems in metal lined chambers enclosed below approximately 6.5 to approximately 8.5 feet through which air flows in portable air purifier units most often placed in lower interior areas, can safely incapacitate biological contaminants below approximately 6.5 to approximately 8.5 feet. Tiny amounts of UV power not enclosed and beamed horizontally or vertically at approximately 6.5 to approximately 8.5 feet and above can safely incapacitate biological contaminants in upper air interior areas.

Aspects provide systems and methods to inhibit and redirect airborne transmission of viruses by preventing the traveling of air containing biological contaminants over distances toward people and time spent by biological contaminants in the air below approximately 6.5 to approximately 8.5 feet before being directed through air purifiers, incapacitated, and/or directed toward upper air UV protected areas.

### BRIEF DESCRIPTION OF THE DRAWINGS/FIGURES

The accompanying drawings, which are incorporated herein and form part of the specification, illustrate aspects and, together with the description, further serve to explain the principles of the aspects and to enable a person skilled in the relevant art(s) to make and use the aspects.

FIG. 1 is a perspective view of a Virus Killing Cyclone™ mechanism (UV air purifier) incorporated into a table according to various aspects.

FIG. 2 is a side view of an Extended Purple Killing Zone™ mechanism (air purifier redirecting air from lower areas to upper areas while being purified with UV) according to various aspects.

FIG. 3 is a top view of a Virus Killing Cyclone™ mechanism (UV air purifier) incorporated into a table according to various aspects.

FIG. 4 is a top view of multiple Virus Killing Cyclones™ mechanisms incorporated into multiple table UV air purifiers in a conference table configuration according to various aspects.

FIG. 5 is a perspective view of an Extended Purple Killing Zone™ mechanism (air purifier redirecting air from lower areas to upper areas while being purified with UV) according to various aspects.

FIG. 6 is a front view of a Virus Killing Cyclone™ mechanism (UV air purifier) incorporated into a countertop according to various aspects.

FIG. 7A is a perspective view of a Social Air Barrier™ mechanism (air purifier creating an air barrier while being purified with UV) according to various aspects.

FIG. 7B is a side view of a Social Air Barrier™ mechanism (air purifier creating an air barrier while being purified with UV) according to various aspects.

FIG. 8A is a perspective view of a Social Air Pillar™ mechanism (air purifier creating an air barrier while being purified with UV) according to various aspects.

FIG. 8B is a side view of a Social Air Pillar™ mechanism (air purifier creating an air barrier while being purified with UV) according to various aspects.

FIG. 9 is an example method for implementing various aspects.

The features and advantages of the aspects will become more apparent from the detail description set forth below when taken in conjunction with the drawings, in which like reference characters identify corresponding elements throughout. In the drawings like reference numbers generally indicate identical, functionally similar, and/or structurally similar elements.

### DETAILED DESCRIPTION

The present invention(s) will now be described in detail with reference to aspects thereof as illustrated in the accompanying drawings. References to “one aspect,” “an aspect,” “an exemplary aspect,” etc., indicate that the aspect described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is submitted that it is within the knowledge of one skilled in the art to affect such feature, structure, or characteristic in connection with other aspects whether or not explicitly described.

The following examples are illustrative, but not limiting, of the present aspects. Other suitable modifications and adaptations of the variety of conditions and parameters normally encountered in the field, and which would be apparent to those skilled in the art, are within the spirit and scope of the disclosure.

Aspects provide an air purification and protection system that can include one or more of a Virus Killing Cyclone™ mechanism, an Extended Purple Killing Zone™ mechanism, a Social Air Barrier™ mechanism, and a Social Air Pillar™ mechanism. All can utilize a Purple Killing Zone™ (the space approximately 6.5 to approximately 8.5 feet high and above in an enclosed space which utilizes ultraviolet radiation (UV) for additional incapacitation of biologic contaminates present in the air). Each of the Virus Killing Cyclone™ mechanism, Extended Purple Killing Zone™ mechanism, Social Air Barrier™ mechanism and Social Air Pillar™ mechanism can utilize an air purifying UV generator to emit UV radiation to incapacitate biologic contaminates present in the air. The UV generator can be a UV light bulb and can emit UVA, UVB, or UVC radiation. In an aspect, the UV generator can emit UVC radiation at a wavelength of 253.7 nanometers.

A Purple Killing Zone™ is the interior space approximately 6.5 to approximately 8.5 feet high and above in an enclosed space which utilizes UV for additional incapacitation of biologic contaminates present in the air which have been directed to a Purple Killing Zone™ by one or all of the Virus Killing Cyclone™ mechanism, Extended Purple Killing Zone™ mechanism, Social Air Barrier™ mechanism, and Social Air Pillar™ mechanism. The Purple Killing Zone™ in any enclosed space can have a beam(s) of UV radiation radiating in one or many directions to incapacitate biological contaminates that flow through it in the upper air of an enclosed space. The Purple Killing Zone™ UV radiation is not enclosed and does not bend lower than approximately 6.5 to approximately 8.5 feet.

The Virus Killing Cyclone™ mechanism can both purify air with UV radiation and/or discharge UV purified air in a generally vertical direction to reach approximately 6.5 to approximately 8.5 feet (and above) in interior areas. For example, the Virus Killing Cyclone™ mechanism can discharge the air to an upper air UV radiation area referred to hereafter as the Purple Killing Zone™. This constant, generally vertical direction of UV air with incapacitated biological contaminants can create a fast-moving air barrier, which can protect people from air discharged from others (normal respiration, coughs, sneezes, etc.) that can include biological contaminants. The Virus Killing Cyclone™ can redirect this potentially virus infected away from potentially infecting people to the air upward to the Purple Killing Zone™.

According to aspects, an air purifier can be situated to intake air containing biological contaminants and can exhaust the air after exposing the biological contaminants to UV radiation. For example, the Virus Killing Cyclone™ mechanism can be incorporated into furniture, such as a table, a desk, or seating. In another example, the Virus Killing Cyclone™ mechanism air purifier can be detached and positioned under and within various surfaces, e.g., a countertop. Another example provides a Virus Killing Cyclone™ mechanism air purifier incorporated into an elevated storage unit, such as a workstation storage cabinet or other similar enclosure. In this way, the Virus Killing Cyclone™ mechanism can be incorporated in a variety of enclosed spaces, for example, restrooms, office spaces, kitchens, bathrooms, and fitness areas. Moreover, the Virus Killing Cyclone™ mechanism air purifier can be incorporated, attached, or retrofitted into desks, conference tables, workstation surfaces, workstation storage cabinets, workstation mobile file boxes, kitchen counters, dining room tables, reception counters, retail counters, fast food counters, government counters such as the Motor Vehicle Administration, bank counters (counters of any kind) coffee tables, ottomans, reclining arm chairs, bus seating, air plane seating, and/or any furniture and/or similar enclosure to be utilized in an enclosed space.

One or more vents can intake air that can then be circulated in the interior of the Virus Killing Cyclone™ mechanism air purifier to expose biological contaminants to UV radiation and incapacitate the biological contaminants. The Virus Killing Cyclone™ can have one or more fans and can facilitate and direct air flow through a UV radiation chamber, which can be partially surrounded by metal or other protective material. The UV radiation chamber does not restrict the air flow past the UV generator and through the exhaust vents. The air flow can include an intake air flow, an interior air flow, and an exhaust air flow. In the interior of the Virus Killing Cyclone™, the interior air flow can being exposed to UV radiation generated, for example, by one or more UV generators positioned in a recessed chamber. The method of the Virus Killing Cyclone™ mechanism can include one or more fans suctioning a large volume of air that might contain biological contaminants. After exposing the biological contaminants to UV radiation and incapacitating the biological contaminants therein, the Virus Killing Cyclone™ can direct the airflow upward to the Purple Killing Zone™. In this way, if a person proximate to the Virus Killing Cyclone™ mechanism has a contagious disease that can be transmitted through particulates in the air, the air containing the particulates can be suctioned into the Virus Killing Cyclone™ mechanism (e.g., that is located in a table) from one or more sides through the intake vents and/or pushed upward to the Purple Killing Zone™ by the exhaust from the vents on the exhaust side of the air purifier to reduce or eliminate airborne transmission. Because these particulates can be transmitted in the air over distances (e.g., six feet) and can remain suspended for extended periods of time, exposing biological contaminants to UV radiation and incapacitating the biological contaminants and/or redirecting them to the Purple Killing Zone™ having upper air UV radiation and for additional exposure to UV radiation, is especially important. Each time the air moves through UV radiation, biological contaminants can be further incapacitated.

Once exhausted by the Virus Killing Cyclone™ mechanism, the air with the incapacitating biological contaminants can be directed generally upward vertically to a height of approximately 6.5 feet to approximately 8.5 feet or more to the Purple Killing Zone™, which will expose the biological contaminants to UV radiation and incapacitate any additional biological contaminants in the air that may have been drawn upward. Because biological contaminants are increasingly incapacitated the longer they are exposed to UV radiation, the Purple Killing Zone™ can include additional and/or larger UV generators, for example, of a greater length and intensity, which can beam large amounts of UV radiation through which the air can be exposed to further incapacitate additional biological contaminants. Accordingly, the UV radiation utilization in the Virus Killing Cyclone™ mechanism proximate to people who might discharge virus-containing particulates and redirecting of the air to the overhead Purple Killing Zone™ can be effective in the additional reduction of biological contaminants in the air.

Aspects provide the Extended Purple Killing Zone™ mechanism air purifier, which can utilize a vertical enclosed chamber design. The Extended Purple Killing Zone™ mechanism can be open at the bottom and the top to facilitate airflow. The Extended Purple Killing Zone™ mechanism can be held in place with adjustable legs at the bottom and top to anchor the Extended Purple Killing Zone™ mechanism against the floor and ceiling of an enclosed space. The Extended Purple Killing Zone™ mechanism can provide lower air suction through vents by one or more fans, which can expose the intake air containing biological contaminants to UV radiation generated by one or more UV generators inside the Extended Purple Killing Zone™ mechanism. The Extended Purple Killing Zone™ mechanism can exhaust air, for example using one or more additional fans. The exhaust air can then flow into the Purple Kill Zone™ and/or additional upper air UV generators installed at the top of the Extended Purple Killing Zone™. As described herein, the Extended Purple Killing Zone™ mechanism can intake air from lower interior areas and provide additional air purification via UV radiation in the Extended Purple Killing Zone™ mechanism by utilizing additional upper air UV radiation attached to the upper portion of the Extended Purple Killing Zone™ mechanism or by utilizing existing UV radiation in the Purple Killing Zone™ (approximately 6.5 to approximately 8.5 feet and above in interior areas).

In other aspects, a Social Air Barrier™ mechanism can utilize the Virus Killing Cyclone™ mechanism in a portable air barrier environment. In the portable air barrier, air can be directed upward to the Purple Kill Zone™ at any location. For example, the Social Air Barrier™ mechanism can be placed on conference tables, floors, the corners of workstations, an edge of an indoor sports field (e.g., at one or more points, or spanning end to end) and can form a protective vertical air barrier that can clean air from both sides of the barrier, for example by exposing the air to UV radiation and sending it upwards to the Purple Killing Zone™. A metal shield inside the barrier can prevent eyes and skin from being exposed to UV, e.g., by preventing a line of sight into any of the side intake vent(s) and or the top exhaust vent(s). An aspect, the barrier can be metal or other protective material. The Social Air Barrier™ mechanism can be made of lightweight plastic, high strength metal, or carbon fiber depending on the area of use. The Social Air Barrier™ mechanism can be adaptable to the area of use. In an example, in an automobile garage, the Social Air Barrier™ mechanism can be driven over as it sits on the floor to protect automotive workers by incapacitating biological contaminants.

In additional aspects, a Social Air Pillar™ mechanism can utilize the Virus Killing Cyclone™ mechanism and can facilitate cleaning of air from all sides. For example, the Social Air Pillar™ mechanism can be circular and/or square and or other shape required to utilize the Virus Killing Cyclone™ mechanism and intake air from all sides.

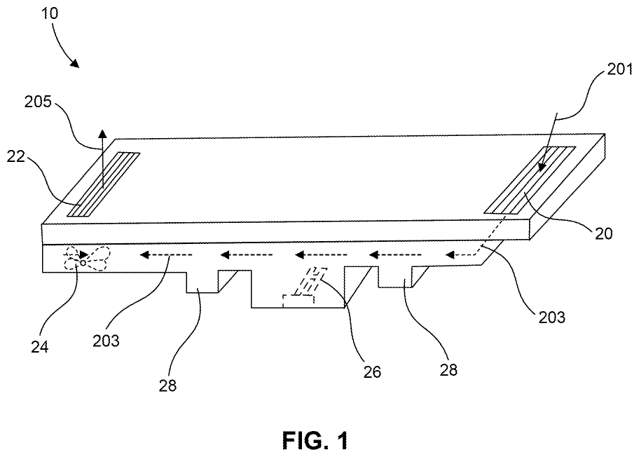

A table 10 incorporating a Virus Killing Cyclone™ mechanism is shown in FIG. 1. In an aspect, the Virus Killing Cycle mechanism can be positioned below the top surface of table 10. In some aspects, the table can be a desk, such as in an office or school setting. In an aspect, the Virus Killing Cyclone™ mechanism air purifier is integral with table 10. In another aspect, the Virus Killing Cyclone™ mechanism air purifier is retrofitted onto an existing table 10. In another aspect, the Virus Killing Cyclone™ mechanism air purifier can be removable from table 10. The Virus Killing Cyclone™ mechanism air purifier in table 10 can include a lower air intake vent 20, a lower air exhaust vent 22, one or more lower air fan(s) 24, one or more lower air UV generators 26, and one or more cleanable gutters 28 to collect and prevent liquids, food and other materials from entering the vents and damaging the UV generator.

In some aspects, the one or more gutters 28 can be recessed channels on either side of lower UV generator 26 that are cleanable. In this way, any liquids or other debris that fall into either the lower air intake vent 20 or the lower air exhaust vent 22 can be retained in and cleaned from gutters 28 instead of flowing to and potentially damaging the UV generator 26. Gutters 28 can include a switch such that opening the cleanable gutters or any opening of the Virus Killing Cyclone™ mechanism can immediately switch off the UV generator.

According to aspects, lower air intake vent 20 can intake air proximate to table 10. In an aspect, a person sitting at table 10 can be proximate to lower air intake vent 20 such that the air taken in by lower air intake vent 20 includes particles from the person, e.g., droplets from their body (e.g., nose, mouth, etc.) These particles can include biological contaminants that can be harmful to others. Accordingly, lower air intake vent 20 proximate to the person can intake the air containing biological contaminants to reduce or prevent dispersion and airborne transmission. In an aspect, lower air intake vent 20 can be positioned at an edge of table 10 to be proximate to a person sitting at table 10 (i.e., lower air intake vent 20 on table 10 can be proximate to a person at seated level).

Lower air that flows through the Virus Killing Cyclone™ mechanism air purifier can include an intake air flow 201 through lower air intake vent 20, an interior air flow 203 through an enclosed chamber, the middle of which is enclosed and lined with protective material such as metal where the air is exposed to UV radiation, and exhausted as exhaust air flow 205 through lower air exhaust vent 22. Air can be rapidly taken in via intake air flow 201 directed by the one or more lower air fan(s) 24. In an aspect, the Virus Killing Cyclone™ mechanism lower air fan(s) 24 can be powerful enough to create an intake air flow 201 capable of suctioning a larger volume of air that might contain biological contaminants. Accordingly, air containing particles from a person sitting at table 10 can be almost completely taken in, exposed to UV radiation and exhausted upward to the Purple Killing Zone™ by the Virus Killing Cyclone™ mechanism.

Interior air flow 203 can be directed through the interior of the Virus Killing Cyclone™ mechanism air purifier by lower air fan(s) 24. Intake air flow 201 can follow a generally vertical path during intake. After intake, interior air flow 203 can follow a path generally parallel to the top surface of table 10 in the interior of the Virus Killing Cyclone™ mechanism air purifier. The interior can include a recessed portion of the chamber. In an aspect, the recessed portion can be lined with a protective material, such as metal. The chamber can contain one or more lower UV generator(s) 26. UV generator 26 can be hidden from view when looking into either lower air intake vent 20 or lower air exhaust vent 22 to maintain eye and skin safety. In some aspects, two or more lower UV generators 26 can be utilized. In some aspects, UV generator 26 can be a 7.5 watt UV light bulb. As the air moves past lower air UV generator 26, the air can be exposed to UV radiation, which can induce damage to the genomes of bacteria, protozoa, and viruses, breaking bonds and forming photodimeric lesions in nucleic acids, DNA, and. RNA and to prevent both transcription and replication and ultimately lead to inactivation of the biological contaminants. In some aspects, fan 24 can operate at a lower speed to slow interior air flow 203 and cause the air to be exposed to UV radiation for a longer duration. In other aspects, lower air fan(s) 24 can operate at a higher speed to create a constant, generally vertical direction of air that has passed through UV radiation with the biological contaminants having been incapacitated, creating a fast-moving air barrier which protects people from air discharge from people (normal respiration, coughs, sneezes, etc.) that can include biological contaminants, and can redirect this potentially virus infected air upward to the Purple Killing Zone™.

Once the air moves past UV radiation created by UV generator 26, lower air fan(s) 24 can create the exhaust air flow 205 to direct the air that has passed through UV radiation with the biological contaminants having been incapacitated, creating a fast-moving air barrier which protects people from air discharge from people (normal respiration, coughs, sneezes, etc.) that can include biological contaminants, and can redirect this potentially virus infected air upward to the Purple Killing Zone™. Exhaust air flow 205 can exit table 10 via lower air exhaust vent 22. At exit, exhaust air flow 205 can follow a generally vertical path that is generally perpendicular to the top surface of table 10. In an aspect, the Virus Killing Cyclone™ mechanism fan 24 can be powerful enough to discharge air such that it can travel upward to a height of approximately 6.5 to approximately 8.5 feet. In this way, instead of dispersing through the lower air in a room, the air can move directly from table 10 to the Purple Killing Zone™ in which existing upper air UV radiation may exist.

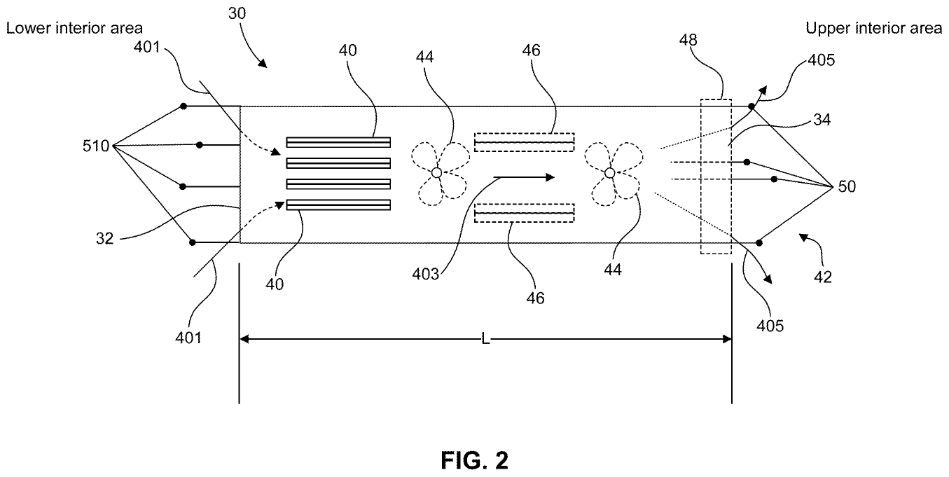

As shown in FIG. 2, the Extended Purple Killing Zone™ mechanism 30 can include one or more lower air intake vent(s) 40, one or more upper exhaust vent(s) 42, one or more upper air fan(s) 44, one or more upper UV generator(s) 46, one or more upper air UV box(es) 48, and one or more spring-loaded legs 50.

In some aspects, the Extended Purple Killing Zone™ mechanism 30 can be a hollow chamber open at both its top and bottom and lined with a protective material, such as metal where UV generator 46 can be placed and can expose air flowing in the interior of the hollow chamber. In some aspects, the length of the Extended Purple Killing Zone™ mechanism 30 can have a length L of approximately 6.5 to approximately 8.5 feet from the lower surface 32 to the upper surface 34. In an aspect, the length of the Extended Purple Killing Zone™ mechanism 30 can be adjustable to retain the Extended Purple Killing Zone™ mechanism between the floor and the ceiling of an interior space. The Extended Purple Killing Zone™ mechanism 30 can include one or more adjustable legs 50 on either end to fit the Extended Purple Killing Zone mechanism 30 in between lower an upper interior air space(s). In an aspect, adjustable legs 50 can be spring-loaded.

Air flow through the Extended Purple Killing Zone mechanism 30 can include an intake air flow 401, an interior air flow 403, and an exhaust air flow 405. In some aspects, upper air fan(s) 44 can be powerful enough to create an intake air flow 401 capable of suctioning a large volume of air that might contain biological contaminants. In some aspects, after intake, upper air fan(s) 44 can direct interior air flow 403 into the interior of the Extended Purple Killing Zone™ mechanism 30. Upper air fan(s) 44 can direct interior air flow 403 from lower air intake vent(s) 40 past UV generator bulb(s) 46. UV generator bulb(s) 46 can be hidden from sight in the hollow chamber lined with a protective material such as metal to prevent eyes and skin from being harmed from UV radiation exposure. In some aspects, two upper UV generator bulbs 46 can be utilized. In some aspects, interior air flow 403 can be directed to upper air UV box(es) 48 for additional UV radiation exposure and, thus, redundant incapacitation of biological contaminants. Accordingly, the air that exits the Extended Purple Killing Zone mechanism 30 into the interior space via passing through UV radiation from upper air UV box(es) 48 and that becomes exhaust air flow 405 that moves through upper exhaust vent 42 can be free of biological contaminants.

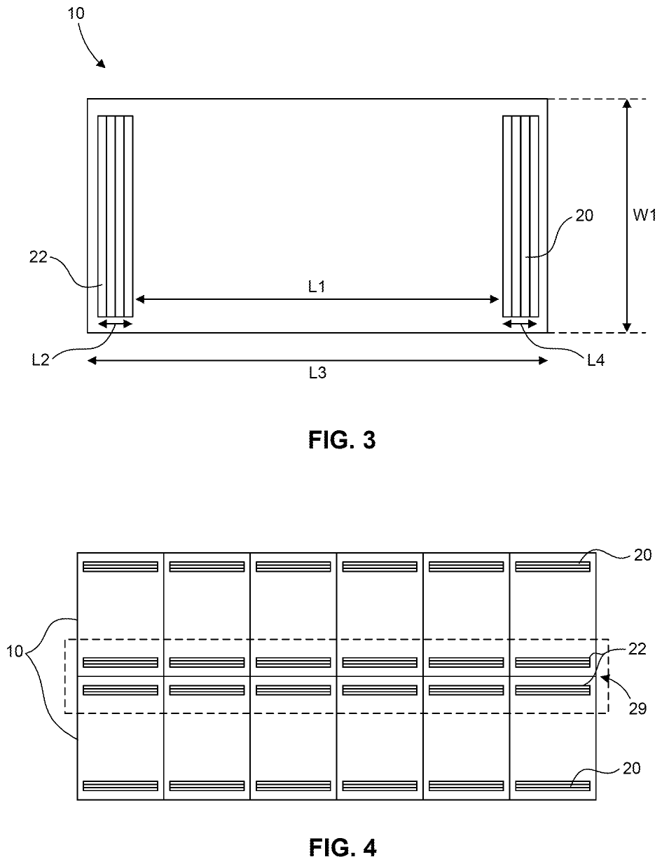

With reference to FIG. 3, table 10 can have a length, L3. L3 can be approximately 24 inches to approximately 36 inches. Table 10 can have a width, W1, of approximately 18 inches to approximately 36 inches. Accordingly, in an aspect, table 10 can be functional as a desk in an office or school setting. The distance between lower air intake vent 20 and lower air exhaust vent 22 can be a length, L1. L1 can be approximately 15 inches to approximately 17 inches. Lower air intake vent 20 can have a length, L4, which can be approximately 2 inches to approximately 4 inches. Similarly, lower air exhaust vent 22 can have a length, L2, which can be approximately 1 inch to approximately 3 inches. In some aspects, lower air exhaust vent 22 is approximately the same length or narrower than lower air intake vent 20 to assure the maximum velocity of the upward air that has had its biological contaminants incapacitated. In an aspect, the width of lower air intake vent 20 and or the width of lower air exhaust vent 22 can be a little narrower than W1 to allow the vents to be supported by a structural framework.

With reference to FIG. 4, in some aspects, multiple tables 10 can be positioned together, such as in an office space cubicle configuration or in a classroom with a row of desks. In other aspects, tables 10 can be nested together to form a conference table. As shown in FIG. 4, tables 10 can be fit together such that exhaust vents 22 can be proximate to each other. In this way, the air that can contain biological contaminants discharged by a person sitting at table 10 can be taken in by the lower air intake vents 20 in table 10 to have the air's biological contaminates exposed to UV radiation and incapacitated and can be prevented from reaching a second person sitting at a second table 10. One or two exhaust vents 22 proximate to each other can form a barrier 29. Barrier 29 can be a shield of fast-moving air that has been treated with UV radiation separating each person sitting at opposite ends of tables 10. The fast-moving air having been treated with UV radiation in tables 10 can move upward in a generally vertical direction to the Purple Killing Zone™, causing all intersecting air containing discharge from people sitting at table 10 to be forced upward for additional exposure to UV radiation and further incapacitation of biological contaminates.

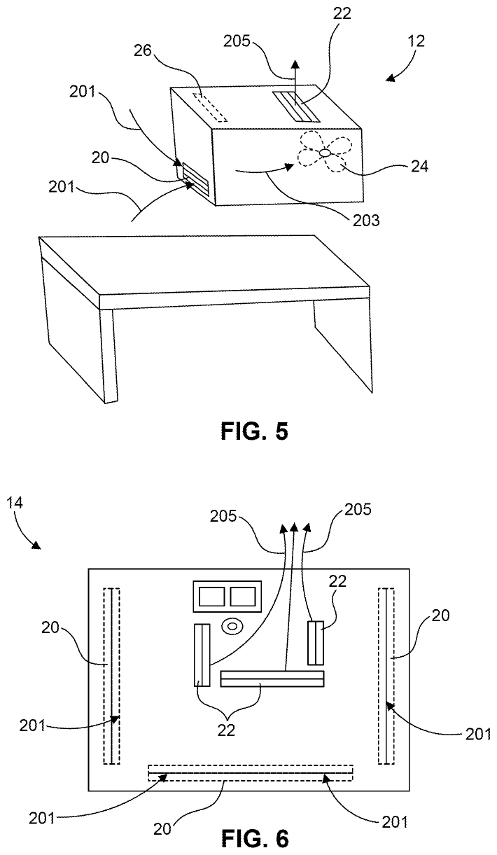

As shown in FIG. 5, in another aspect, a Virus Killing Cyclone™ mechanism can be incorporated into a compartment 12, e.g., a workstation storage cabinet. In an aspect, compartment 12 can be elevated. In this way, lower air intake vent 20 can intake air proximate to people who are in or near a workstation. Because they might be at varying elevations, for example sitting or standing, discharge from people (normal respiration, coughs, sneezes, etc.) at a higher point than if they were sitting can have biological contaminates exposed to UV radiation and incapacitated and redirected upward toward the Purple Killing Zone for additional exposure to UV radiation and further incapacitation of biological contaminates.

Accordingly, compartment 12 can be positioned to quickly intake air containing biological contaminates, expose it to UV radiation, and redirect it upward toward the Purple Killing Zone to prevent the air and biological contaminants from dispersing across the room. In an aspect, UV generator bulb(s) 26 contained in compartment 12 can be positioned in an upper portion such that the UV generator bulb(s) is hidden from view looking through the lower air intake vent 20 and/or the lower air exhaust vent 22. In some aspects, UV generator 26 can be automatically switched off prior to opening the elevated compartment 12, such as to change the UV light bulb(s). For example, compartment 12 can include an automatic switch to turn off UV generator bulb(s) 26 when the Virus Killing Cyclone™ mechanism activated.

Similar to table 10 (FIG. 1), air containing biological contaminates can be suctioned into lower air intake vent 20 by fan 24, exposed to UV generator bulb(s) 26 and redirected upward via lower air exhaust vent 22 toward the Purple Killing Zone™. Lower air fan(s) 24 can be powerful enough to have the air discharged upward in a generally vertical direction at a high rate of speed to reach the overhead Purple Killing Zone™ for additional incapacitation of biological contaminates.

As shown in FIG. 6, in another aspect, a Virus Killing Cyclone™ mechanism can be incorporated into a countertop console 14. It in aspect, a countertop console 14 can be integrated into a kitchen countertop or other large table surface. In this way, lower air intake vent 20 can intake air proximate to people who are working in a kitchen. Because this can be numerous people, exposing and incapacitating biological contaminates with UV radiation air can be especially important. In addition, similar to the previous example, people working in kitchens might be standing and so discharge from people (normal respiration, coughs, sneezes, etc.) can occur at a higher point than if they were sitting at the countertop. The Virus Killing Cyclone™ mechanism enables these biological contaminants to be exposed to UV radiation and incapacitated and redirected upward toward the Purple Killing Zone™. Accordingly, countertop console 14 can be positioned to quickly intake air containing biological contaminates to prevent the air from dispersing across the room. In an aspect, UV generator bulb(s) 26 contained in countertop console 14 can be hidden from sight. Similar to table 10 (FIG. 1), air can have its biological contaminates incapacitated in countertop console 14 by being exposed to UV radiation and then discharged via lower air exhaust vent 22. Lower air fan(s) 24 can be powerful enough to have the air discharged upward in a generally vertical direction at a high rate of speed and/or volume to reach the overhead Purple Killing Zone™ for additional exposure to UV radiation and further incapacitation of its biological contaminates.

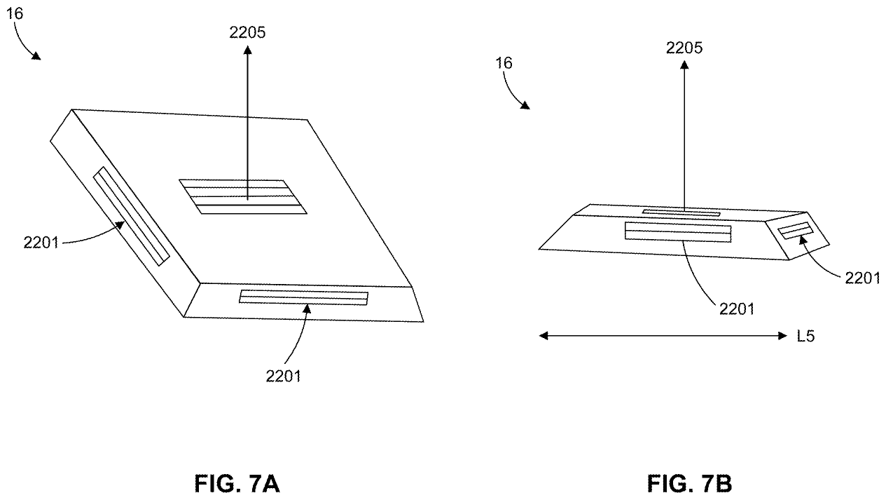

As shown in FIGS. 7A-B, in some aspects, a Virus Killing Cyclone™ mechanism air purifier can be incorporated into a Social Air Barrier™ mechanism 16 in a portable air barrier environment. Air can be rapidly taken in via intake air flow 2201 capable of suctioning a larger volume of air that might contain biological contaminants.

Once the air moves past UV radiation in Social Air Barrier™ mechanism 16, exhaust air flow 2205 can direct the air that has passed through UV radiation with the biological contaminants having been incapacitated upward to the Purple Killing Zone™. At exit, exhaust air flow 2205 can follow a generally vertical path. In the portable air barrier, air can be directed upward to the Purple Kill Zone™ at any location. For example, the Social Air Barrier™ mechanism 16 can be placed on conference tables, floors, the corners of workstations, an edge of a stage during a play or performance or presentation, an edge of an indoor sports field (e.g., at one or more points, or spanning end to end) and can form a protective vertical air barrier that can clean air from both sides of the barrier by exposing the air to UV radiation and sending it upwards to the Purple Killing Zone™. Accordingly, the Social Air Barrier™ mechanism 16 can be positioned at a selected location to quickly intake air containing biological contaminates to prevent the air from dispersing across the room. The air can be exposed to UV radiation, and redirected upward toward the Purple Killing Zone™. A shield made from a protective material, such as metal, can be positioned inside the air barrier and can prevent eyes and skin from being exposed to UV radiation, e.g., by preventing a line of sight into any of the side intake vent(s) and or the top exhaust vent(s).

In some aspects, the Social Air Barrier™ UV radiation 16 can be made of lightweight plastic, high strength metal, and/or carbon fiber. In an aspect, the material composition can depend on the area of use such that the Social Air Barrier™ 16 can be adaptable to the area of use. In an example, in an automobile garage, the Social Air Barrier™ mechanism 16 can be driven over as it sits on the floor to protect automotive workers by incapacitating biological contaminants. In an aspect, the Social Air Barrier™ mechanism 16 can have a length, L5, of approximately 12 inches, approximately 24 inches, or approximately 36 inches.

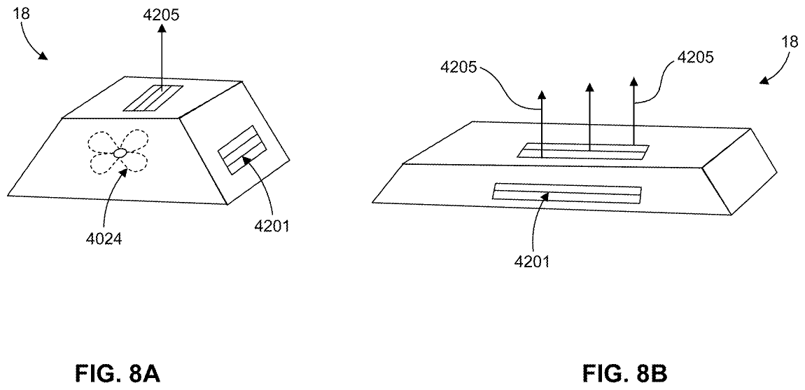

As shown in FIGS. 8A-B, in some aspects, a Social Air Pillar™ mechanism 18 can facilitate cleaning of air from all sides. In some aspects, the Social Air Pillar™ mechanism 18 can be circular and/or square and can pair with Social Air Barrier™ mechanism 18 to direct air flow and enable the Virus Killing Cyclone™ mechanism to intake air from all sides.

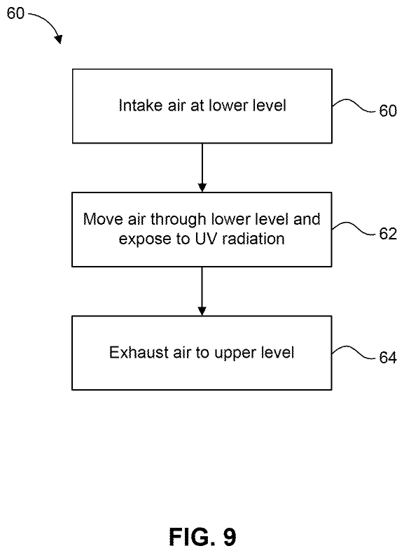

As shown in FIG. 9, an example method 60 can be employed for additional air incapacitation of its biological contaminates using UV radiation and fans. In step 60, a lower level system can intake air for exposure to UV radiation and incapacitation of its biological contaminates using UV radiation and fans. Air flow in the lower level system can include an intake air flow 201, an interior air flow 203, and an exhaust air flow 205. In some aspects, lower level air for incapacitation of its biological contaminates using UV radiation and fans can be performed by a Virus Killing Cyclone™ mechanism contained in table 10 (FIG. 1). In some aspects, Virus Killing Cyclone™ mechanism in table 10 can be incorporated into a conference room table or cubicles in an office space, or in a single desk and/or a row of desks in a school setting. In other aspects, the Virus Killing Cyclone™ mechanism can be incorporated into an elevated compartment 12 (FIG. 5) for when people are standing in an interior space, or a countertop console 14 (FIG. 6) such as in a kitchen area. In the lower level Virus Killing Cyclone™ mechanism system, lower air intake air vent 20 can intake air. Lower air intake vent 20 can be proximate to one or more persons to intake air containing biological contaminates discharged from them. The air proximate to the person can quickly be taken in by the Virus Killing Cyclone™ mechanism to prevent the biological contaminates from dispersing through a room and harming the health of someone else. One or more lower fans 24 can be in a Virus Killing Cyclone™ mechanism. Lower fan(s) 24 can be powerful enough to create an intake air flow 201 capable of suctioning a larger volume of air that might contain biological contaminants, including air proximate to a person.

In step 62, airflow can be moved through a UV chamber and exposed to UV radiation from UV generator bulb(s) 26. UV generator bulb(s) 26 can be contained in a recessed compartment to prevent them from being in the line of sight of and causing harm to anyone's eyes or skin in the proximity of the air purifier (e.g., when looking into either lower air intake vent 20 or lower air exhaust vent 22). Two UV generator bulb(s) 26 can be utilized. Interior air flow 203 can be directed through the interior of the Virus Killing Cyclone™ mechanism by lower air fan(s) 24. As the air moves through UV radiation from UV generator bulb(s) 26, the air can be exposed to UV radiation, which can incapacitate any biological contaminants. Lower air fan(s) 24 can additionally operate at a lower speed to slow interior air flow 203 and cause the air to be exposed to UV radiation for a longer duration.

In step 64, the air with biological contaminants incapacitated can be exhausted to the upper level of a room or area. Once the air with biological contaminants moves through the UV radiation from the UV generator bulb(s) 26, lower air fan(s) 24 can create an exhaust air flow 205 to direct the air with biological contaminants incapacitated upward in a generally vertical direction. Exhaust air flow 205 can exit table 10 via lower air exhaust vent 22. In an aspect, lower air fan(s) 24 can be powerful enough to create an exhaust air flow 205 with biological contaminants incapacitated that can travel upward in a generally vertical direction approximately 6.5 feet to approximately 8.5 feet. In this way, instead of dispersing through the room, the air can move directly from table 10 to the Purple Killing Zone which can have other additional UV radiation.

According to various aspects, an air purification and protection system which incapacitates biological contaminants in interior space as they exist, can be produced and transmitted and can provide a barrier of air, a portion of whose biological contaminants have been incapacitated, to inhibit transmission and provide additional incapacitation of biological contaminants in interior space that is taller than approximately 6.5 to approximately 8.5 feet. The air purification and protection system can include a lower system and an upper system.

According to various aspects, a lower system can be positioned in the lower interior space. The lower system can be incorporated in furniture and other ordinary interior non-organic items including UV light air purifiers whose air flow has been modified with tape to exhausted upward at a perpendicular angle to their intake. The lower system can include a first intake vent to intake air proximate to the lower system, one or more first fans having a powerful suction to facilitate intake of the air and create a first air flow in the lower system, one or more first UV light bulbs producing UV light which incapacitates a portion of the biological contaminants in air moving through the UV light via the first air flow, and a first exhaust vent to exhaust via the first air flow in a generally vertical direction because of the tape modifications, creating an fast moving air barrier which protects people on each side of the air barrier from air discharge from people (normal respiration, coughs, sneezes, etc.) that can include biological contaminants, and can redirect this potentially virus infected air upward to the upper UV radiation system or through an enclosed chamber to be delivered directly through upper air UV radiation beamed horizontally across the upper interior space powerful enough to incapacitate the majority of biological contaminants in the upper interior space at approximately 6.5 feet to approximately 8.5 feet and above.

According to various aspects, an upper system positioned above the lower system in the upper interior space at approximately 6.5 feet to approximately 8.5 feet and above, the upper system comprising UV radiation beamed horizontally across the upper interior space powerful enough to incapacitate the majority of biological contaminants in air moving through the UV radiation. It is to be appreciated that the Detailed Description section, and not the Summary and Abstract sections, is intended to be used to interpret the claims. The Summary and Abstract sections may set forth one or more but not all exemplary aspects of the present invention(s) as contemplated by the inventor(s), and thus, are not intended to limit the present invention and the appended claims in any way.

The present invention(s) have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

What is claimed is:

1. An air purification and protection system in an interior space having a lower interior space and an upper interior space, comprising:
a lower system positioned in the lower interior space, the lower system comprising:
a first intake vent to intake air proximate to the lower system,
a fan to create a first air flow in the lower system,
a UV generator to produce UV radiation to incapacitate biological contaminants in the first air flow, and
a first exhaust vent to exhaust the first air flow in a generally vertical direction; and
an upper system positioned above the lower system in the upper interior space, the upper system to receive the first air flow exhausted from the first exhaust vent, the upper system emitting UV radiation horizontally to incapacitate biological contaminants.

2. The air purification and protection system of claim 1, wherein the lower system is coupled to a table.

3. The air purification and protection system of claim 1, wherein the lower system is portable to produce an air barrier at a selected location.

4. An air purification and protection system in a lower interior space, comprising:
an intake vent to intake air proximate to the system;
one or more fans to intake of the air and create an air flow in the system;
one or more UV generators to produce UV radiation to incapacitate biological contaminants in the air moving through the UV radiation via the air flow; and
an exhaust vent to exhaust the air via the first air flow in a generally vertical direction,
wherein the air flow comprises a fast moving air barrier to prevent dispersion of biological contaminants in the air, and
wherein the system is portable to produce the air barrier at a selected location.
